(19) United States
(12) Patent Application Publication
(10) Pub. No.: US 2006/0177522 A1
(43) Pub. Date: Aug. 10, 2006

(54) Rinsing solution for contact lenses

(75) Inventors: Jean-Claude Yvin, Saint-Malo (FR); Didier Leroy, Pleurtuit (FR); Patrice Rat, Paris (FR); Jean-Michel Warnet, Paris (FR)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: LABORATOIRES GOEMAR S.A.

(21) Appl. No.: 10/523,316

(22) PCT Filed: Jul. 30, 2003
(86) PCT No.: PCT/FR03/02412

§ 371 Date: Feb. 21, 2006

(30) Foreign Application Priority Data
Aug. 2, 2002 (FR) 02/09913

(51) Int. Cl. A61K 33/14

(52) U.S. Cl. 424/676

(57) ABSTRACT

The invention relates to the use of an aqueous ionic solution for rinsing contact lenses, particularly contact lenses made of hydrophilic materials. Said aqueous ionic solution is produced from sea water, has an ionic composition that is of the same quality as that of sea water, and the quality of which is such that said aqueous ionic solution has a pH of 4 to 9, preferably 7 to 8, and an osmolality of 150 to 700 mOsm/kg, preferably 250 to 350 mOsm/kg.

The invention relates to a rinsing solution for contact lenses.

Among the contact lenses to be found on the market, the most used are those which are made of hydrophilic materials.

Between two successive uses, these contact lenses are rinsed using solutions, in particular aqueous which make it possible to disinfect them; overnight, these lenses are stored in humid medium.

Numerous rinsing solutions of this kind exist.

Due to their hydrophilic character, these lenses absorb appreciable quantities of these rinsing solutions, in particular overnight when they are kept immersed in these solutions.

When placed on the eye, they therefore bring the solutions in question onto the ocular surface, in contact with which they can be salted out. It is evident that when they comprise constituents irritating to the eye, the patient wearing the lenses can develop intolerances such that he has to abandon the use of the contact lenses.

This is precisely what is observed in practice; intolerances to contact lenses are increasingly numerous and a certain percentage of these intolerances is very probably due to the presence of the irritant constituents just called into question.

The purpose of the invention is therefore especially to remedy the drawbacks of the prior art and to make available to the user a rinsing solution for contact lenses comprising no constituents irritating to the eye.

The Applicant company has found that a solution corresponding to the desiderata outlined above is an aqueous ionic solution obtained from sea water the ionic composition of which is qualitatively that of sea water and quantitatively such that on the one hand its pH is from 4 to 9, preferably from 7 to 8 and that on the other hand its osmolality is from 150 to 700, preferably from 250 to 350 m Osm/kg.

More particularly, it is an aqueous ionic solution obtained from sea water characterized by:
a pH value preferably lower than or at most equal to the lowest pH values of sea water,
an osmolality lower than that of sea water and
a composition which from an ionic point of view is qualitatively and quantitatively that of sea water, with the exception that from the qualitative point of view, on the one hand, the potassium concentration is higher than that of sea water and, on the other hand, the Na, Mg, Ca and Cl concentrations are lower than those of sea water, said concentrations being
for Na*, from 1300 to 1500, preferably from 500 to 1000 mg/l,

for K*, from 4500 to 6500, preferably from 5000 to 6000 mg/l,

for Mg**, from 50 to 1300, preferably from 100 to 500 mg/l,

for Ca**, from 20 to 350, preferably from 40 to 200 mg/l,

for Cl*, from 4000 to 6000, preferably from 4500 to 5000 mg/l.

The aqueous ionic solution in question forms the subject of the French Patent Application No. 99 16814 filed on 31st Dec. 1999.

The invention therefore relates to the use, as a rinsing solution for contact lenses, of the abovementioned aqueous ionic solution.

The superiority of the solution used according to the invention with respect to the rinsing solutions for contact lenses which already exist was demonstrated by the “in vitro” tests described hereafter.

These tests were carried out using cells originating from a human conjunctival cell line known as Wong Kilbourn Derivated Conjunctival Cell (WKD, ATTCC 20.2).

These cells were brought into 100% contact, i.e. immersed, for at least 15 minutes on the one hand in the solution used according to the invention and on the other hand in four commercial contact-lens rinsing solutions marketed under the trademarks “Complete”, “SoloCare”, “Opti Free” and “Renu” respectively by the Allergan, Ciba Vision, Alcon and Bausch and Lomb laboratories.

The effects produced on these conjunctival cells were evaluated by different tests known as “MIFALC” (Microtitration Fluorimetric Assays on living cells) in which what are called fluorescent “probes” are used, applied directly to adherent living cells, namely

the neutral red test

the Hoechst 33342 or HO/Propidium Iodide or PI test

the H2DCF-DA test

the hydroethydine test

the Rhodamine 123 test

the Nonyl Acridine Orange (NAO) test

which, respectively make it possible to know the effect of the solutions tested on the following cell markers:

cell viability,

chromatin condensation (apoptosis),

reactive oxygen species,

superoxide anion,

mitochondrial transmembrane potential,

mitochondrial mass

The Wong Kilbourn Derivated Conjunctival Cell cells (WKD, ATCC 20.2) are cultured in 75 cm² flasks in an incubator thermostatically controlled at 37° C. under a humid atmosphere containing 5% of CO₂.

The culture medium used is DMEM enriched to 10% with SVK, containing 1% glutamine and antibiotics, in particular ampicillin/Kanamycin.

The culture medium is replaced every 2-3 days, the cells are trypsinated at 80% confluence i.e. once a week on average.

The tests are carried out in 96-well microplates. 24 hours before treatment of the cells tested using the different rinsing solutions, the 96-well microplates are seeded with the WKD cells at a rate of 5,000 cells per well.

One plate per test is used, i.e. 8 plates in total.

Given that the fluorescent probes emit in the UV range, it is necessary to use special microplates with black edges (in particular in the Hoechst 33342-Ho/IP test).

24 hours after the seeding, the microplates are treated with the solutions to be tested.

The incubation lasts 15 minutes and is carried out according to the following plate diagram comprising the columns 1, 2, 3, 4, 5, 6, 7, 8, 9, 10, 11 and 12 and rows A, B, C, D, E, F, G, and H.

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | | Con | Con | Con | Con | Con | Con | Con | Con | Con | Con | |
| C | | Sero | Sero | Sero | Sero | Sero | Sero | Sero | Sero | Sero | Sero | |
| D | | Comp | Comp | Comp | Comp | Comp | Comp | Comp | Comp | Comp | Comp | |
| E | | Opti | Opti | Opti | Opti | Opti | Opti | Opti | Opti | Opti | Opti | |
| F | | Renu | Renu | Renu | Renu | Renu | Renu | Renu | Renu | Renu | Renu | |
| G | | Solo | Solo | Solo | Solo | Solo | Solo | Solo | Solo | Solo | Solo | |
| H | | | | | | | | | | | | |

in which the different abbreviations signify:
Con: control=BSS Comp: Complete Renu: Renu
Sero: Solution used according to the invention Opti: Optifree
Solo: Solo care

The solutions are used pure and under sterile conditions.

The experiment is repeated three times consecutively with this same protocol.

In the different tests, the effects obtained are expressed by a more or less intense fluorescence.

A description is given hereafter and in a first phase of the manner in which the different MIFALC tests referred to above are implemented.

1. Neutral Red Test

In this test, the effect of the tested solutions on cell viability and membrane integrity is evaluated, the results being expressed in % of fluorescence with respect to the control.

These results can be analyzed using the “SigmaStat 32” statistics software version 2.0 (Analysis of variance and post hoc test, DUNNET Test).

The Neutral Red test is carried out according to Borenfreund's protocol (which provides for 50 μml/3 h); this protocol has been used and validated in different European multicentric studies; it is considered as one of the most sensitive for evaluation of the cytotoxicity of a substance.

Neutral Red is a vital inclusion dye the binding of which inside cells is a function of cell viability and membrane integrity.

It thus makes it possible to evaluate the cytotoxic effect of a chemical or physical agent after 24 to 72 hours, on average, of contact.

The result is expressed in standard fashion as a percentage of viability with respect to the non-treated control.

The Neutral Red test result is evaluated by fluorimetric detection in order to increase the specificity of the reaction according to a MiFALC (Microtitration Fluorimetric Assays on Living Cells) procedure.

The sensitivity of detection of Neutral Red under these conditions is 50 pg/ml. (Rat P. et al., Cell Biol. Toxicol., 1994, 10, 329-337; Rat P. et al., Methods in Enzymology, 1995, 252,331-340.).

2. Hoechst 33342/Propidium Iodide Test

By this test, the chromatin condensation (apoptosis), in other words the cell death mechanism

(Apoptosis/Necrosis->Hoechst/Neutral Red ratio)

is evaluated.

The “Hoechst 33342” used is a probe specific to adenine and thymine, which are constituents of the DNA of cells.

Counter-labelling with propidium iodide guarantees a binding of “Hoechst” on normal cells or in apoptosis only, as the nuclei or the DNA debris of cells in necrosis are very rapidly stained by the intercalating propidium iodide which then prevents the subsequent binding of “Hoechst”.

A dark blue fluorescence is observed after binding of “Hoechst” on the DNA of the normal cells.

If the number of normal cells reduces, the fluorescence of the “Hoechst 33342” decreases.

But chromatin condensation associated with an apoptosis phenomenon induces an overexpression of the fluorescence of “Hoechst” and therefore an increase in fluorescence of the signal/control.

It is thus possible to observe, after only 15 minutes of treatment with the OptiFree solution, a steady reduction in the signal.

On the contrary, the fluorescence signal remains high and even increases in relation to the control for SOLOCARE AND RENU solutions.

It would be possible to deduce from this the hypothesis of an apoptosis mechanism in the cytotoxicity of these products on our retained cell model.

3. DCFH-DA (or H₂DCF-DA) and Hydroethidine Test

These tests make it possible to evaluate a possible radical-like production or more precisely the synthesis of hydrogen peroxide or that of superoxide anions respectively.

The DCFH-DA or hydroethydine fluorogenic probes emit a green fluorescence on contact with free radicals (ROS, H₂O, superoxide anion).

This very labile radical-like production is very sensitive to the artefacts of standard evaluation protocols (trypsination, monodispersion of the cells . . . .), hence the importance of using cold light cytofluorimetric technology (MCCM), which makes it possible to use these probes directly on adherent living cells without trypsination and without monodispersion of the cells.

The limitation of these artefacts makes it possible to considerably improve the quality of the signal and to be able to work, with a high level of sensitivity (pg-fg/ml), on standard cells (fibroblasts, tenocytes, conjunctival cells) which have a lower base radical-like level than specialized cells (macrophages or polynuclear cells in suspension) used in flow cytometry.

More particularly, the DCFH-DA test allows the evaluation of the reactive oxygen species, principally hydrogen peroxide.

The H2 DCF-DA probe is cleaved inside the cells by cellular esterases.

The fluorescein derivative formed, H2DCF, is not fluorescent, but can become fluorescent on contact with reactive oxygen species (for example hydrogen peroxide) in order to form dichlorofluorescein which emits a green fluorescence.

It is thus possible to observe an overproduction of free radicals as a function of incubation time (15, 30, 45, 60 minutes) or as a function of the different products tested.

As regards the HydroEthidine test it will be noted that the latter, which is not fluorescent, is converted to fluorescent ethidine (ethidium bromide) under the effect of superoxide anions.

This specific reaction of the superoxide anion makes it possible to demonstrate the role of this radical in certain cytotoxicity mechanisms.

4. Rhodamine 123 and NAO Tests

These tests make it possible to evaluate the activity and the total mitochondrial mass.

The mitochondrion seems to be an early and privileged target in the cytotoxicity mechanism of numerous molecules.

Up to now, the evaluation of mitochondrial activity has been difficult and required very cumbersome equipment (microelectrodes or CMF laser).

The new methods of Microtitration Cytofluorimetry (MCM), which incorporate cold light fluorimetry technology (MCCM), allow the use of specific probes directly on living cells.

More particularly, the Rhodamine 123 (Rh 123) test is a well-known probe in flow cytometry (CMIF) for its specific binding to the mitochondrion.

The Rhodamine 123 (Rh 123) fluorescent probe is therefore applied to living cells in order to evaluate the variations in the total mitochondrial activity following the action of the solutions tested.

The fluorescence of the Rhodamine 123 is proportional to the mitochondrial transmembrane potential.

The Rhodamine 123 is evaluated with fluorimetric detection (Exc. 490 nn,/Em. 535 nm) according to a MiFALC (Microtitration Fluorimetric Assays on Living Cells) procedure.

The sensitivity of detection of the Rhodamine 123 under these conditions is 20 pg/ml (Rat P. et al., Cell Biol. Toxicol., 1994. 10, 329-3). The interpretation of the results requires analysis of an associated NAO (Nonyl Acridine Orange) test.

5. Nonyl Acridine Orange Test

Nonyl Acridine Orange is a red-fluorescent probe the fluorescence of which is independent of the transmembrane potential.

In fact, the NAO binds to the cardiolipid of the mitochondrial membrane. The greater the mitochondrial mass and surface, the greater will be the fixation of the NAO and the higher its fluorescence.

During cell stress, the mitochondria can increase or reduce in size indirectly inducing a modification of the mitochondrial transmembrane potential.

Therefore, analysis of the Rhodamine 123 and NAO tests allows fine evaluation of the mitochondrial activity (Rhodamine 123 test) which can be weighted by the modifications of the mitochondrial mass (NAO test) following cell stress.

In other words, it makes it possible to have a precise idea of the effect produced by the solutions tested.

As has just been seen, the retained tests give rise to fluorescence signals.

It is therefore necessary to evaluate these fluorescence signals.

In order to do this, a Microtitration Cytofluorimetry (MCM) technique incorporating cold light fluorimetry technology (MCCM: Microplate Cold light Cytometry) is used, described by Rat P. et al., in Methods in Enzymology, 1995. 252, 331-340.

The detection limit of this technique is: pico-femtogram of probe/ml.

The detection spectrum is: 280 to 870 nm.

It is thus possible to carry out intracellular fluorescent probe measurements directly in microplates on living cells (MIFALC tests: Microtitration Fluorimetric Assay on Living Cells).

Thanks to this technique, it becomes possible to study short-term and long-term cytotoxicity as well as kinetics continuously, as the cell line used has enzymatic equipment which is stable over time which is different with respect to what occurs in the primoculture; moreover the technology used allows the evaluation of the fluorescence kinetics on adherent cells.

It also makes it possible to evaluate the cytotoxicity directly on adherent living cells (MIFALC—Microtitration Fluorimetric Assays on Live Cells tests), as the fact of trypsinating or monodispersing the cells in order to allow their use in immunochemistry or in flow cytrometry induces different artefacts which can alter the observation and the correct interpretation of certain labile markers (for example free radicals . . . .).

The cells of the eye are adherent cells which lose certain intracellular functions if they are separated from their support or if they are dissociated from one another.

It is therefore preferable to work on adherent living cells using the MiFALC procedures.

Finally, the use of a cytofluorimetric technology on microplates has the advantage of combining the detection specificity of the fluorimetric methods, the sensitivity of cold light technology (pico-femtog/ml of detected probe) and the reproducibility of methods on microplates.

Interpretation of the test results just described makes it possible to

evaluate the type of cell death induced (necrosis or apoptosis),

evaluate the radical oxygen species (H₂0₂) induced,

evaluate a possible overproduction of superoxide anion and

evaluate the intensity of the mitochondrial activity.

a) Evaluation of the Type of Cell Death (Necrosis-Apoptosis)

Combination of several fluorescent labels makes it possible to simultaneously measure necrosis and apoptosis-type cell death mechanisms.

In order to do this, a combination of a probe such as Neutral Red for measuring membrane integrity with two DNA probes, Hoechst 33342 and Propidium iodide, for measuring apoptosis and chromatin condensation mechanisms was used.

A significant increase in the “Hoechst” in relation to the control is generally the sign of chromatin condensation during apoptosis mechanisms which is moreover confirmed by fluorescence microscope observations.

It can thus be observed that the solution used according to the invention is globally inert and does not produce any particular cytotoxicity whatever for necrosis or apoptosis.

On the contrary, commercial solutions such as Renu® or Solo care® induce a significant increase in the fluorescence of “Hoechst”, a sign of an apoptosis-type cell stress.

For Optifree®, in less than 15 minutes, necrosis-type cell death is observed; this necrosis can in fact be an apopto-necrosis mechanism which could be made more specific with additional tests.

In the case of the product Complete®, cell stress is observed with increase of the two markers the mechanism of which can be clarified by the other tests.

Therefore, as from this first series of tests, two groups are observed, on the one hand the solution used according to the invention which is globally inert on the conjunctival cells of the human eye, and, on the other hand, the different commercial multi-functional products for rinsing contact lenses which all induce a necrosis-, apopto-necrosis- or apoptosis-type cell stress.

b) Evaluation of the Radical-Like Forms of Oxygen (1120)

Appearance kinetics of radical-like forms of oxygen are observed.

These kinetics correspond to the tests carried out with the H2DCF-DA probe, which reacts with the different radical-like forms of oxygen, and particularly with hydrogen peroxide (H₂O₂).

Extremely different behaviour is thus observed between the solution according to the invention and the commercial products.

After contact for 15 minutes, there is no significant difference between the different known commercial solutions.

There does not therefore seem to be any specific role of the radical-like forms of oxygen or radical oxygen species in inducing the cell stresses previously observed over short periods of 15 minutes.

On the contrary, the triggering of these apoptosis and necrosis mechanisms can induce modifications in the production of radical forms.

Thus, after contact for 60 minutes, all the commercial solutions induce a reduction in the radical-like forms of oxygen probably linked to a cell-stress effect and even to a reduction in the number of cells, due to the cell deaths observed as from the first 15 minutes (necrosis and apoptosis) seen previously with the Neutral Red and “Hoechst” tests.

In the case of the solution used according to the invention, it is observed that the cells are functional and react to this exogenous agent by a radical overproduction without inducing any significant alteration of the cell as the overall cytotoxicity tests (Neutral Red tests) are not disturbed.

c) Evaluation of an Overproduction of Superoxide Anion

An overproduction of superoxide anion is observed following treatment with the different solutions tested.

In fact it is observed, that there is no significant difference in relation to the control and no overproduction of superoxide anion is observed whatever the products. It should be noted that the production of superoxide anion in the cells incubated with the solution used according to the invention has a tendency to be the lowest of the products tested without observing any significant difference with respect to the large standard deviations.

Overall, considerable cell stress is therefore observed tending towards cell death by apoptosis but without being linked to a significant radical-like stress.

Additional tests on the mitochondrion make it possible to explore other apoptosis-induction routes.

d) Evaluation of Mitochondrial Activity

For this evaluation, two fluorescent probes are simultaneously combined.

The first of these probes namely the Rhodamine 123 probe, makes it possible to evaluate the mitochondrial transmembrane potential, while the second, NAO (nonyl acridine orange), makes it possible to measure the mitochondrial mass.

Thus, after contact for only 15 minutes, a significant reduction is observed in the mitochondrial transmembrane potential for the commercial solutions of Renu® or Solocare® type.

It is known that such a reduction in the mitochondrial transmembrane potential appears in early phases of apoptosis which were observed as soon as from 15 minutes and is confirmed by the appearance of chromatin condensation with the probes such as the “Hoechst”.

Moreover, dissociation of the mitochondrial mass and of the mitochondrial activity is observed for commercial solutions such as Complete® and Optifree® which confirms the cell stress observed previously.

The solution used according to the invention itself appears totally inert compared with other commercial solutions used for rinsing contact lenses.

Overall, it therefore appears that all the commercial products induce significant cell stress.

Unlike the solution used according to the invention, Solocare® and Renu induce, as soon as from 15 minutes of incubation, very distinct apoptosis mechanisms with chromatin condensation and alteration of the mitochondrial functions.

The solutions Optifree® and Complete induce cell stress characterized by the triggering of the mitochondrial compensation system, reduction of the mass, increase in the mitochondrial transmembrane potential, which, if the stress persists, can lead to apoptosis-necrosis mechanisms.

All of the preceding statements and conclusions show very clearly the superiority, by virtue of its harmlessness, of the solution used according to the invention in comparison with the already existing solutions for rinsing contact lenses.

This applies all the more as the stresses observed and described above, which make it possible to lead to these conclusions were observed after contact for only 15 minutes with the solutions in question. In practice, these solutions can adsorbe themselves on the lens during rinsing and be salted out over several hours after being brought into contact with the patient's eyeball.

From a practical point of view, the aqueous ionic solutions, to the extent that they are intended for use for rinsing contact lenses can be prepared as indicated in the patent application identified above in which useful details as to their properties are also to be found.

1. Use for rinsing contact lenses, in particular those made of hydrophilic materials, of an aqueous ionic solution obtained from sea water the ionic composition of which from the qualitative point of view is that of sea water and from the quantitative point of view is such that on the one hand its pH is from 4 to 9, preferably from 7 to 8 and that on the other hand its osmolality is from 150 to 700, preferably from 250 to 350 m Osm/kg.

2. Use for rinsing contact lenses, in particular those made of hydrophilic materials, of an aqueous ionic solution, characterized by:
a pH value preferably lower than or at most equal to the lowest pH values of sea water,
an osmolality lower than that of sea water and
a composition from the ionic point of view which is qualitatively and quantitatively that of sea water, with the exception that from the qualitative point of view, on the one hand, the potassium concentration is higher than that of sea water and, on the other hand, the Na, Mg, Ca and Cl concentrations are lower than those of sea water, said concentrations being
for Na*, from 1300 to 1500, preferably from 500 to 1000 mg/l,
for K*, from 4500 to 6500, preferably from 5000 to 6000 mg/l,
for Mg**, from 50 to 1300, preferably from 100 to 500 mg/l,
for Ca**, from 20 to 350, preferably from 40 to 200 mg/l, for Cl*, from 4000 to 6000, preferably from 4500 to 5000 mg/l.
